(19) United States
(12) Patent Application Publication
(10) Pub. No.: US 2012/0115215 A1
(43) Pub. Date: May 10, 2012

(54) LIGHT SOURCE REFLECTOR

(75) Inventor: Robert D. Eckles, Malcolm, NE (US)

(73) Assignee: LI-COR, Inc., Lincoln, NE (US)

(21) Appl. No.: 12/939,335

(22) Filed: Nov. 4, 2010

(51) Int. Cl. C12M 1/34; F21V 7/00

(52) U.S. Cl. 435/288.7; 362/297

(57) ABSTRACT

A light source reflector includes a structure having three or more internal reflective surfaces defining a first light entry aperture and a second light exit aperture having an area that is greater than the area of the entry aperture. A single light source, or a plurality of uniformly distributed light sources, such as one or more LEDs, located proximal the entry aperture provides illumination, which impinges on the reflective surfaces from the entry aperture and is reflected off the surfaces to provides a substantially uniform illumination pattern exiting the exit aperture. The reflector advantageously allows for the use of a source (e.g., LED(s)) that is smaller than the targeted area of illumination and also avoids creating hot-spots as the reflector does not focus the light, but rather reflects mirror images of the source off of the reflective surfaces.

### BACKGROUND

The present invention relates generally to light reflectors, and more particularly to light source reflectors for use in photosynthesis measurement and analysis systems.

In photosynthesis measurement and analysis systems it is useful to provide a uniform illumination pattern on a subject under investigation, such as a leaf, within a controlled analysis chamber. Light sources can be expensive and hence providing illumination uniformity at low costs can be an issue, especially as the target size requiring illumination increases.

Therefore it is desirable to provide light sources that overcome the above and other problems.

### BRIEF SUMMARY

The present invention provides light sources, and in particular light source reflectors for use in photosynthesis measurement and analysis systems.

A light source reflector includes a structure having three or more internal reflective surfaces defining a first light entry aperture and a second light exit aperture having an area that is greater than the area of the entry aperture. A single light source, or a plurality of uniformly distributed light sources, such as one or more LEDs, located proximal the entry aperture provides illumination, which impinges on the reflective surfaces from the entry aperture and is reflected off the surfaces to provides a substantially uniform illumination pattern exiting the exit aperture. The reflector advantageously allows for the use of a source (e.g., LED(s)) that is smaller than the targeted area of illumination and also avoids creating hot-spots as the reflector does not focus the light, but rather reflects mirror images of the source off of the reflective surfaces.

According to one embodiment, a light source reflector is provided that typically includes a light reflecting structure having three or more reflective surfaces coupled around a central axis and defining a first aperture and a second aperture, wherein the first aperture has a first area and wherein the second aperture has a second area that is the same as or greater than the first area. The light source reflector also typically includes one or more light emitting elements positioned proximal to the first aperture, wherein illumination emitted by the light emitting elements is reflected by the three or more surfaces such as to provide a substantially uniform illumination pattern exiting the second aperture. In certain aspects, each of the three or more surfaces extends from the first aperture to the second aperture at an angle relative to the axis of greater than about 5 degrees such that the second area is larger than the first area. In certain aspects, the one or more light emitting elements include one or more light emitting diodes (LEDs), or other light sources, that are arranged and spaced substantially uniformly proximal to, or within, the first area defined by the first aperture.

According to another embodiment, a light source reflector is provided that typically includes a light reflecting structure having three or more reflective surfaces coupled around a central axis and defining a first aperture and a second aperture, wherein the first aperture has a first area and wherein the second aperture has a second area that is the same as or greater than the first area. The light source reflector also typically includes one or a plurality of light emitting elements positioned proximal to the first aperture, wherein illumination emitted by the light emitting elements is reflected by the three or more surfaces such as to provide substantially uniform illumination proximal to the second aperture. In certain aspects, each of the three or more surfaces extends from the first aperture to the second aperture at an angle relative to the axis of greater than about 5 degrees such that the second area is larger than the first area. In certain aspects, the one or a plurality of light emitting elements include one or more LEDs, or other light sources, that are arranged and spaced substantially uniformly proximal to, or within, the first area defined by the first aperture.

According to yet another embodiment, a light source for use in measuring photosynthesis is provided. The light source typically includes one or a plurality of light emitting elements, and a light reflecting structure having three or more reflective surfaces coupled around a central axis and defining a first aperture and a second aperture, wherein the first aperture has a first area and wherein the second aperture has a second area that is the same as or greater than the first area. In certain aspects, the one or a plurality of light emitting elements are located proximal to the first aperture, wherein illumination emitted by the light emitting elements is reflected by the three or more surfaces such as to provide a substantially uniform illumination pattern exiting the second aperture. In certain aspects, each of the three or more surfaces extends from the first aperture to the second aperture at an angle relative to the axis of greater than about 5 degrees such that the second area is larger than the first area. In certain aspects, the one or a plurality of light emitting elements include one or more LEDs, or other light sources, that are arranged and spaced substantially uniformly proximal to, or within, the first area defined by the first aperture.

According to a further embodiment, a chamber for measuring photosynthesis is provided. The chamber typically includes a light reflecting structure having three or more reflective surfaces coupled around a central axis and defining a first aperture and a second aperture, wherein the first aperture has a first area and wherein the second aperture has a second area that is the same as or greater than the first area, and one or a plurality of light emitting elements positioned proximal to the first aperture, wherein illumination emitted by the light emitting elements is reflected by the three or more surfaces such as to provide a substantially uniform illumination pattern exiting the second aperture. In certain aspects, each of the three or more surfaces extends from the first aperture to the second aperture at an angle relative to the axis of greater than about 5 degrees such that the second area is larger than the first area. In certain aspects, the one or a plurality of light emitting elements include one or more LEDs, or other light sources, that are arranged and spaced substantially uniformly proximal to, or within, the first area defined by the first aperture.

According to yet a further embodiment, a light source reflector for use in the measurement of photosynthesis and leaf chlorophyll fluorescence is provided. The Reflector typically includes a light reflecting structure having three or more reflective surfaces coupled around a central axis and defining a first aperture and a second aperture, wherein the first aperture has a first area and wherein the second aperture has a second area that is the same as or greater than the first area. The reflector also typically includes first and second light emitting elements positioned proximal to the first aperture, wherein the first element emits at a first wavelength different than a second wavelength emitted by the second emitter, wherein illumination emitted by the first and second light emitting elements is reflected by the three or more surfaces such as to provide a substantially uniform illumination pattern of both the first wavelength and the second wavelength exiting the second aperture.

According to still a further embodiment, a leaf chlorophyll fluorescence chamber is provided for measuring chlorophyll fluorescence. The chamber typically includes a light reflecting structure having three or more reflective surfaces coupled around a central axis and defining a first aperture and a second aperture, wherein the first aperture has a first area and wherein the second aperture has a second area that is the same as or greater than the first area, and one or a plurality of light emitting elements positioned proximal to the first aperture, wherein illumination emitted by the light emitting elements is reflected by the three or more surfaces such as to provide a substantially uniform illumination pattern exiting the second aperture.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1a illustrates a front view of a light source reflector 10 according to one embodiment, FIG. 1b shows a cross-sectional side view of reflector 10, FIG. 1c shows a front isometric view of reflector 10, and FIG. 1d shows a rear isometric view of reflector 10.

FIG. 2 illustrates a top view of a light source reflector having a plurality of light sources positioned proximal to the light entry aperture.

FIG. 3 illustrates a Fluorometer light source according to one embodiment.

FIG. 4 illustrates a side view of two prototype light source reflector structures made of aluminum, with polished aluminum interior reflective surfaces.

### DETAILED DESCRIPTION OF THE INVENTION

The present invention provides light source reflectors. The light source reflectors of the various embodiments described herein are particularly useful as light sources in photosynthesis measurement and analysis systems.

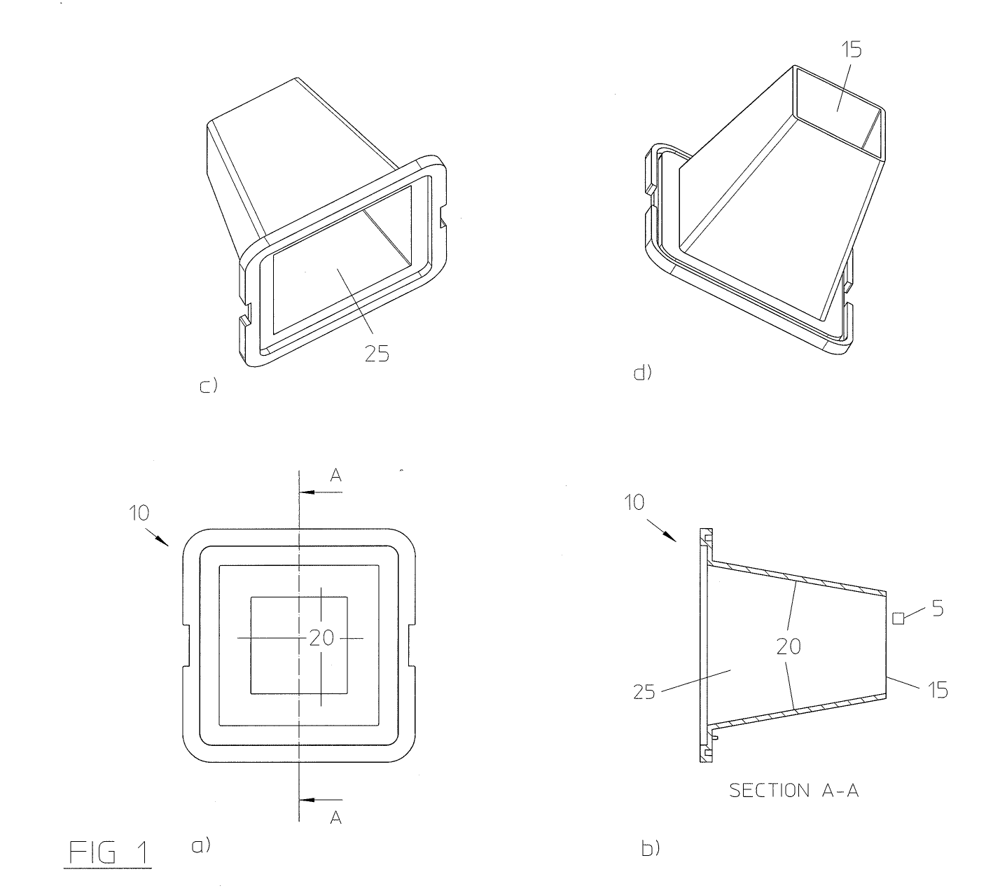

FIG. 1 illustrates a light source reflector 10 according to one embodiment. FIG. 1a shows a front view of reflector 10, FIG. 1b shows a cross-sectional side view of reflector 10, FIG 1c shows a front isometric view of reflector 10, and FIG. 1d shows a rear isometric view of reflector 10. As shown, reflector 10 includes a structure having four (4) walls defining interior linear reflective surfaces 20 and coupled together to form a square-conical-type interior structure having square-shaped apertures, with the interior surfaces 20 being angled such that aperture 15 has an area that is larger than the area of aperture 25. For example, as shown in FIG. 1b, the walls have a 10° angle with respect to a central axis (and hence a total 20° divergence as indicated) such that the area defined by aperture 15 is smaller than the area defined by aperture 25. In certain embodiments, the angle the walls make with the central axis ranges from about 2° to about 45° or preferably from about 5° to about 30° or a bit larger.

It should be understood that, in certain embodiments, the exterior walls of the structure themselves need not be linear, or parallel to the interior reflective surfaces 20, but rather can have bulk shapes, or varied external shapes. It is desirable, however, that the interior reflective surfaces 20 defined by the walls be linear (e.g., no curved surfaces) so as to avoid focusing light that could create hot spots or non-uniformities. For example, reflector 10 may have a structure that appears circular on the exterior (i.e., square in a circle cross section with a circular-conical-shaped exterior), but with linear interior reflective surfaces 20 as shown in FIG. 1. Similarly, reflector 10 may have a structure that appears like a square or rectangular block on the exterior, or a cylindrical block on the exterior, but with linear reflective surfaces 20 angled with respect to a central axis a shown in FIG. 1b.

In one embodiment, the walls are formed of a metal material, for example a reflective metal material such as aluminum. The internal surface of a wall that defines a surface 20 may be polished to further enhance reflectivity. Other useful metallic materials include, for example, Aluminum, Brass, Copper, and Gold. Further, in certain embodiments, the walls of the structure can be made of multiple materials. For example a substrate material may be used for the bulk of a wall, and a second material can be attached to the bulk material to form the reflective surface 20. The second material may be attached to the substrate material by way of adhesion (e.g., glue or other adhesive material), welded on, deposited on or otherwise formed on the substrate material, directly or on an intermediary material. In one embodiment, for example, a substrate material includes a metal or a plastic material and a reflective surface is formed by depositing vacuum metalized aluminum thereon. Examples of plastic substrate materials include ABS (Acrylonitrile butadiene styrene), Polycarbonate, acrylic, and polystyrene, and examples of useful reflective materials include Aluminum, Gold, Silver, etc. Plastic materials are useful in certain embodiments to help control characteristics of the reflector, such as temperature.

In certain embodiments, reflector 10 includes three or more interior reflective surfaces 20. With three surfaces 20, apertures 15 and 25 will have a triangular shape. With more than four surfaces 20, the apertures may have different polygonal shapes (e.g., pentagonal, hexagonal, octagonal, decagonal, etc.) depending on the number of surfaces 20. It should also be understood that aperture 15 need not have the same shape or geometry as aperture 25. For example, aperture 15 may have a shape of an equilateral triangle and aperture 25 may have a shape of an isosceles triangle or a right triangle. In such embodiments, the interior surfaces 20 may have different angles with respect to the central axis and/or a surface 20 may include a non-linearity (e.g., a portion of a surface 20 may have one angle with respect to the central axis and another portion of the same surface 20 may have a different angle with respect to the central axis). Additionally, an entire surface 20 or a portion of a surface 20 may have a surface normal that does not point to the central axis from a center point of the surface (contrast this with an embodiment where, for example, the apertures are both equilateral triangles—the normals to all three surfaces 20 necessarily point to the same central axis from the central point of each surface). For embodiments having apertures with different geometries, constraints may be needed to achieve uniformity for devices having large asymmetries of the apertures,. Such constraints might include using an even number of reflective surfaces, using bilateral symmetry in the angular distribution of the reflective surfaces and/or in the placement of the light emitting elements.

In one embodiment, one or a plurality of light emitting elements 5 (single element shown) are placed proximal to the entry aperture, e.g., aperture 15. A single light emitting element may include a single LED, or other light source emitting at the desired wavelength(s). Similarly, a plurality of light emitting elements may include a plurality of individual LEDs, or an LED light tile with two or more LEDs having the same or different light emitting characteristics, or a plurality of other light source types emitting at the desired wavelength(s). An example of useful wavelength ranges might include red light from 620 nanometer to 680 nanometer wavelengths and blue light from 430 nanometer to 470 nanometer wavelengths for chlorophyll fluorescence and photosynthesis applications. Illumination emitted by the light emitting element(s) is reflected by the surfaces 20 such as to provide a substantially uniform illumination pattern exiting the second aperture 25. In certain aspects, the plurality of light emitting elements are arranged and spaced substantially uniformly within and proximal to the area defined by the aperture 15. The light emitting elements can be placed at or below (e.g., outside of structure 10) the plane defined by aperture 15 such that light emitted thereby enters aperture 15 and is reflected by surfaces 20 so as to provide substantially uniform illumination proximal to the second aperture. For example, light from the sources proximal to aperture 15 impinging on reflective surfaces 20 results in mirror images of the sources from all four surfaces 20 exiting the aperture 25.

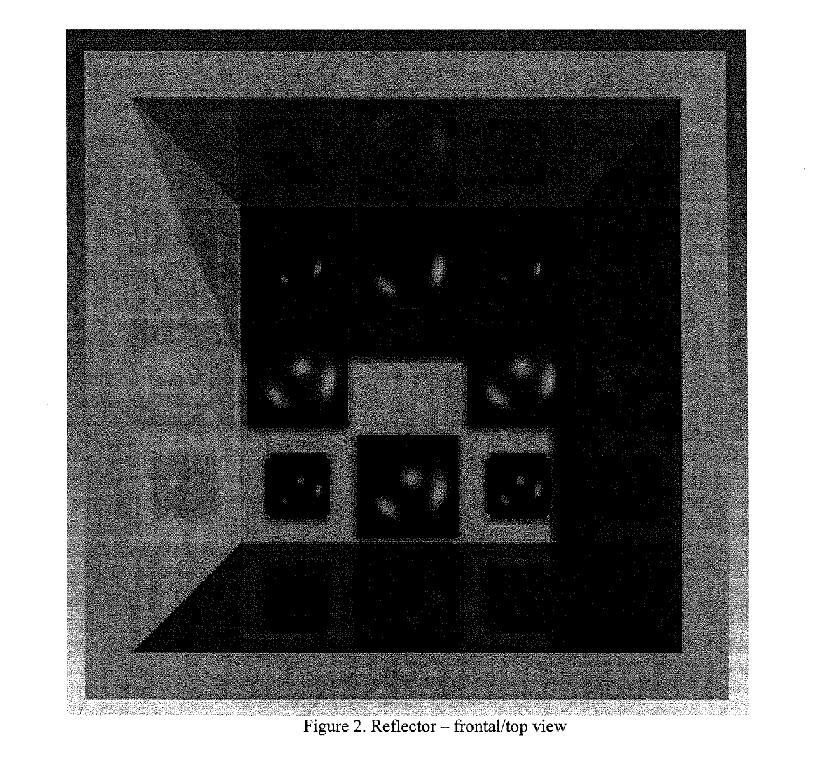

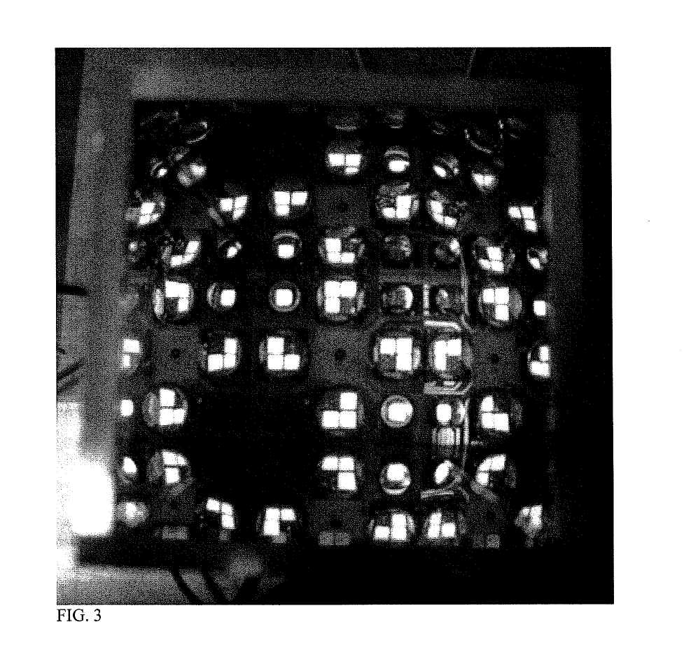

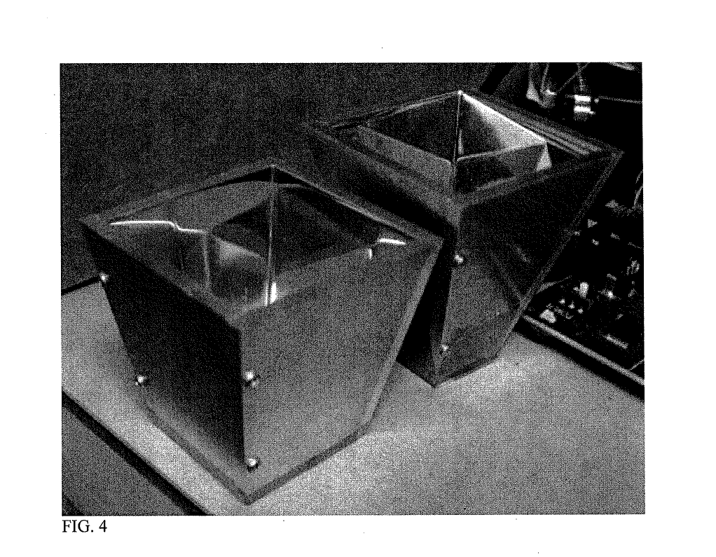

FIG. 2 illustrates a top view of a light source reflector having a plurality of light sources positioned proximal to the light entry aperture (e.g., aperture 15). As shown, the plurality of light sources includes different sized light sources arranged and spaced substantially uniformly within the area defined by the light entry aperture. FIG. 3 illustrates a Fluorometer light source. FIG. 3 is an example of an illuminator with a combination of 2 or more different colored LED emitters, e.g., for the purpose of measuring leaf chlorophyll fluorescence. Such a reflector advantageously allows for mixing different colors of emitters in a uniform fashion. Such a reflector can advantageously be used as a light source for the (near-)simultaneous measurement of photosynthesis and leaf chlorophyll fluorescence. FIG. 4 illustrates a side view of two prototype light source reflector structures made of bulk aluminum, with polished aluminum interior reflective surfaces.

In certain embodiments, light source reflectors as described herein are particularly useful in photosynthesis measurement and analysis systems. For example, a light source reflector 10 can be used in, or as part of, a photosynthesis measurement chamber and/or in a chlorophyll fluorescence measurement system. Light entering the entry aperture 15 is reflected off of the surfaces 20 and exits aperture 25 so as to provide uniform illumination on a sample (e.g., leaf) located proximal to exit aperture 25. As above, embodiments described herein advantageously avoid focusing light and hence avoid creating “hot spots” and allow for the use of a light source that is smaller than the targeted area of illumination while concomitantly providing a high uniformity of illumination at the targeted area. One or more detectors (not shown; e.g., light detectors/fluorescence detectors and/or gas analysis and measurement detectors) may be appropriately located proximal to the second aperture to detect various characteristics of a target being illuminated depending on the application. For example, one or more light detectors may be positioned proximal a chamber holding a leaf for use in leaf chlorophyll fluorescence measurement applications, and/or a gas analysis system be positioned proximal to, or coupled with, a chamber for detecting changes in CO₂ and/or H₂O in photosynthesis and transpiration measurement applications.

Experiments on uniformity of red saturating/actinic light were performed on the reflector prototypes of FIG. 3 using a 4×6 detection grid proximal to the exit aperture. The results, using arbitrary scale, show good uniformity as follows:

### Data Collected

| | | | | | |
|---|---|---|---|---|---|
| 182 | 180 | 185 | 188 | 187 | 187 |
| 183 | 179 | 183 | 186 | 183 | 187 |
| 179 | 180 | 183 | 185 | 185 | 183 |
| 178 | 181 | 184 | 189 | 189 | 189 |

### Uniformity Statistics

| | |
|---|---|
| AVG: | 183.9583333 |
| MAX: | 189 |
| MIN: | 178 |
| STD DEV: | 3.368384552 |
| MAX DEV: | 5.041666667 |
| DEV PERCENT (From | 2.741% |
| Avg) | |

Using various embodiments as described herein, it is possible to provide a uniform illumination on a target having a photon flux density of greater than about 20,000 μmol/(m·sec).

While the invention has been described by way of example and in terms of the specific embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A light source reflector, comprising:
a light reflecting structure having three or more reflective surfaces coupled around a central axis and defining a first aperture and a second aperture, wherein the first aperture has a first area and wherein the second aperture has a second area that is the same as or greater than the first area; and
one or more light emitting elements positioned proximal to the first aperture, wherein illumination emitted by the light emitting elements is reflected by the three or more surfaces such as to provide a substantially uniform illumination pattern exiting the second aperture.

2. The light source reflector of claim 1, wherein each of the three or more surfaces extends from the first aperture to the second aperture at an angle relative to the axis of greater than about 5 degrees such that the second area is larger than the first area.

3. The light source reflector of claim 1, wherein the one or more light emitting elements includes a plurality of LEDs.

4. The light source reflector of claim 1, wherein the one or more light emitting elements are arranged and spaced substantially uniformly proximal to the first area defined by the first aperture.

5. The light source reflector of claim 1, wherein a cross section of the light reflecting structure, perpendicular to the axis, has a polygonal shape.

6. The light source reflector of claim 5, wherein the polygonal shape is selected from a group of shapes consisting of triangular, square, hexagonal, and octagonal.

7. The light source reflector of claim 1, wherein the reflective surfaces comprise a polished metal.

8. The light source reflector of claim 7, wherein the polished metal is polished aluminum.

9. The light source reflector of claim 1, wherein the reflective surfaces comprise a substrate material coated with a reflective material.

10. The light source reflector of claim 9, wherein the substrate material includes a plastic material.

11. The light source reflector of claim 9, wherein the reflective material includes vacuum metalized aluminum.

12. The light source reflector of claim 1, wherein the geometry defining the first area of the aperture is different than the geometry defining the second area of the second aperture.

13. A light source for use in measuring photosynthesis, comprising:
one or a plurality of light emitting elements; and
a light reflecting structure having three or more reflective surfaces coupled around a central axis and defining a first aperture and a second aperture, wherein the first aperture has a first area and wherein the second aperture has a second area that is the same as or greater than the first area; and
wherein the one or a plurality of light emitting elements are located proximal to the first aperture, wherein illumination emitted by the light emitting elements is reflected by the three or more surfaces such as to provide a substantially uniform illumination pattern exiting the second aperture.

14. The light source of claim 13, wherein each of the three or more surfaces extends from the first aperture to the second aperture at an angle relative to the axis of greater than about 5 degrees such that the second area is larger than the first area.

15. The light source of claim 13, wherein the one or a plurality of light emitting elements includes a plurality of LEDs that are arranged and spaced substantially uniformly proximal to the first area defined by the first aperture.

16. A chamber for measuring photosynthesis, the chamber comprising:
a light reflecting structure having three or more reflective surfaces coupled around a central axis and defining a first aperture and a second aperture, wherein the first aperture has a first area and wherein the second aperture has a second area that is the same as or greater than the first area; and
one or a plurality of light emitting elements positioned proximal to the first aperture, wherein illumination emitted by the light emitting elements is reflected by the three or more surfaces such as to provide a substantially uniform illumination pattern exiting the second aperture.

17. The chamber of claim 16, wherein each of the three or more surfaces extends from the first aperture to the second aperture at an angle relative to the axis of greater than about 5 degrees such that the second area is larger than the first area.

18. The chamber of claim 16, wherein the one or a plurality of light emitting elements include a plurality of LEDs that are arranged and spaced substantially uniformly proximal to the first area defined by the first aperture.

19. A light source reflector for use in the measurement of photosynthesis and leaf chlorophyll fluorescence, comprising:
a light reflecting structure having three or more reflective surfaces coupled around a central axis and defining a first aperture and a second aperture, wherein the first aperture has a first area and wherein the second aperture has a second area that is the same as or greater than the first area; and
first and second light emitting elements positioned proximal to the first aperture, wherein the first element emits at a first wavelength different than a second wavelength emitted by the second emitter, wherein illumination emitted by the first and second light emitting elements is reflected by the three or more surfaces such as to provide a substantially uniform illumination pattern of both the first wavelength and the second wavelength exiting the second aperture.

20. A leaf chlorophyll fluorescence chamber for measuring chlorophyll fluorescence, the chamber comprising:
a light reflecting structure having three or more reflective surfaces coupled around a central axis and defining a first aperture and a second aperture, wherein the first aperture has a first area and wherein the second aperture has a second area that is the same as or greater than the first area; and
one or a plurality of light emitting elements positioned proximal to the first aperture, wherein illumination emitted by the light emitting elements is reflected by the three or more surfaces such as to provide a substantially uniform illumination pattern exiting the second aperture.

21. The chamber of claim 20, further comprising one or more light detectors for measuring light emitting characteristics of a sample placed proximal to the second aperture.
